(19) United States
(12) Patent Application Publication
(10) Pub. No.: US 2025/0228753 A1
(43) Pub. Date: Jul. 17, 2025

(54) METHOD OF PRODUCING SOLID PARTICLE

(71) Applicant: KAO CORPORATION, Tokyo (JP)

(72) Inventors: Ryoichi HARUKI, Chuo-ku (JP); Saki HAMADA, Chuo-ku (JP); Hayato MIYAZAKI, Chuo-ku (JP); Nobuhiro NONAKA, Chuo-ku (JP)

(73) Assignee: KAO CORPORATION, Tokyo (JP)

(21) Appl. No.: 18/414,907

(22) Filed: Jan. 17, 2024

(51) Int. Cl. A61K 8/11; A61K 8/25; A61Q 1/02

(52) U.S. Cl. CPC A61K 8/11; A61K 8/25; A61Q 1/02; A61K 2800/43; A61K 2800/651; A61K 2800/87

(57) ABSTRACT

Provided is a method of producing a solid particle including the following: heating a raw material composition containing an oily component to impart fluidity thereto; granulating the raw material composition having imparted thereto the fluidity to provide a granular raw material; and bringing the granular raw material and powder into contact with each other under a state in which vibration is applied to the powder to coat the surface of the granular raw material with the powder.

### FIELD OF THE INVENTION

The present invention relates to a method of producing a solid particle and a solid particle.

### BACKGROUND OF THE INVENTION

Solid particles that have heretofore been known such as solid cosmetics have been generally in such a form that powder is subjected to compression molding to be stored in a shallow-bottom tray like a foundation or in such a form that a composition that is a solid at room temperature is molded into a predetermined shape like a lipstick. In addition to the solid cosmetics in such forms, a solid cosmetic in a granular shape (granular solid cosmetic) has been proposed in recent years.

In, for example, Patent Literature 1, there is a description that a granular solid cosmetic shaped into a granular shape having an average particle diameter of from 1 mm to 5 mm is obtained by a method including: dropping a liquefied cosmetic into a liquid oil having no compatibility with the cosmetic to mold the cosmetic into a granular shape; reducing the temperature of the cosmetic molded into the granular shape to solidify the cosmetic; and then separating the cosmetic from the liquid oil.

### PRIOR ART DOCUMENTS

### Patent Literature

Patent Literature 1: Japanese Patent Application Laid-open No. 2009-274974

### SUMMARY OF THE INVENTION

The liquid oil is used in a production process for the granular solid cosmetic as described in Patent Literature 1. Accordingly, unless the liquid oil is sufficiently separated, the liquid oil remains on the surface of the granular solid cosmetic. Owing to the foregoing, the granular solid cosmetic is liable to adhere to a container or the particles of the granular solid cosmetic are liable to be bonded to each other, and hence the adhesion resistance of the cosmetic reduces. Further, when a solid cosmetic such as a lipstick is merely granulated, the cosmetic is weak against impact involved in its transportation or the like, and hence easily deforms and coalesces. In addition, when the hardness of the solid cosmetic is increased, the cosmetic can resist the transportation or the like, but its sense of use is impaired owing to the hardness.

The present invention relates to a method of producing a solid particle that is excellent in transportation resistance while satisfying adhesion resistance and a sense of use. The term “adhesion resistance” means that adhesion between the solid particle and a container, and adhesion and bonding between the solid particles are suppressed.

The inventors of the present invention have found that a solid particle that is excellent in transportation resistance while satisfying adhesion resistance and a sense of use is obtained by: granulating a raw material composition containing an oily component having imparted thereto fluidity by heating; bringing the granulated raw material composition and powder into contact with each other; and coating the surface of the granular raw material with the powder while applying vibration to the powder at the time of the contact.

The present invention relates to the following items [1] and [2].

[1] A method of producing a solid particle, comprising the following: heating a raw material composition containing an oily component to impart fluidity thereto; granulating the raw material composition having imparted thereto the fluidity to provide a granular raw material; and bringing the granular raw material and powder into contact with each other under a state in which vibration is applied to the powder to coat a surface of the granular raw material with the powder.

[2] A solid particle having a core-shell structure, comprising: a core portion formed of a raw material composition; and a shell portion configured to coat at least part of a surface of the core portion, wherein the shell portion is formed of the raw material composition having incorporated thereinto powder, and the raw material composition having incorporated thereinto the powder has a thickness of 110 μm or more.

According to the production method according to at least one embodiment of the present invention, the solid particle that is excellent in transportation resistance while satisfying adhesion resistance and a sense of use can be provided.

### BRIEF DESCRIPTION OF DRAWINGS

FIG. 1 is a schematic view for illustrating a method of producing a solid particle including using a vibration feeder according to at least one embodiment of the present invention.

FIG. 2 is a schematic view for illustrating a method of producing a solid particle including using a bowl feeder according to at least one embodiment of the present invention.

FIG. 3 is a photograph for showing a result obtained by observing a section of a solid particle according to at least one embodiment of the present invention with an electron microscope.

FIG. 4 is a photograph for showing a result obtained by observing a section of a solid particle of each of Comparative Examples with an electron microscope.

### DETAILED DESCRIPTION OF THE INVENTION

The present invention is described below on the basis of its exemplary embodiments with reference to the drawings. The present invention relates to a method of producing a solid particle.

### [Solid Particle]

A solid particle according to at least one embodiment of the present invention is a solid particle having a core-shell structure, the particle including: a core portion formed of a solid raw material composition that is granular; and a shell portion configured to coat at least part of the surface of the core portion. The shell portion is formed of a layer of the raw material composition having incorporated thereinto powder. The powder is incorporated into the raw material composition in a thickness of, for example, about 100 μm from the surface of the core portion, though the thickness varies depending on the properties of the powder and conditions for the production thereof. The solid particle according to at least one embodiment of the present invention is preferably produced by a method of producing a solid particle according to at least one embodiment of the present invention to be described later.

In the solid particle according to at least one embodiment of the present invention, the strength of the inside of the solid particle near its surface is improved by the adhesion of the powder and its incorporation into the raw material composition. Accordingly, it may be possible to suppress the crush of the solid particle at the time of its transportation due to contact between the particles or between the particle and a container. Thus, the exposure of the core portion is suppressed, and adhesion between the solid particles can be suppressed. Accordingly, the solid particle may be excellent in transportation resistance. Further, a reduction in sense of use can be suppressed because the powder is present near the surface.

The term “solid particle” refers to a particle that is a solid at room temperature (25° C.), and has such a property as to soften or melt to obtain fluidity when heated to a temperature higher than room temperature, for example, 50° C. or more. A particle obtained by subjecting powder to compression molding is excluded from the solid particle as used in the present invention. To obtain such solid particle, as described later, for example, a raw material composition having a melting point of 50° C. or more only needs to be used.

The term “particle” comprehends not only a spherical shape or a substantially spherical shape but also a spheroidal shape, a substantially spheroidal shape, a flat shape, a spindle shape, a polyhedral shape, a fibrous shape, or an indefinite shape. Among them, a spherical shape, a substantially spherical shape, a spheroidal shape, or a substantially spheroidal shape is preferred because of the following reasons: a contact area between the particles is small, and hence the adhesion resistance and transportation resistance of the particle are improved; and the appearance thereof is satisfactory. Further, the term comprehends, for example, a shape imitating some letter or symbol, and shapes imitating characters, such as a person, an animal, and a thing. The solid particle according to at least one embodiment of the present invention may be one kind of those shapes, or may be a combination of two or more kinds thereof.

### [Shape of Solid Particle]

Although the size of the solid particle is not particularly limited, the average projected area of the particles when the particles are mounted on a plane is preferably 0.5 mm² or more, more preferably 1 mm² or more, still more preferably 1.5 mm² or more from the viewpoints of, for example, ease of use such as the ease with which the particles are taken in a hand, the difficulty with which the particles roll, the ease with which the particles are collapsed, a design property, and ease of production. In addition, the average projected area of the solid particles is preferably 320 mm² or less, more preferably 80 mm² or less, still more preferably 20 mm² or less in terms of, for example, ease of use such as the ease with which the particles are taken in a hand, the ease with which the particles are collapsed, and a design property. In particular, the average projected area of the solid particles is preferably 0.5 mm² or more and 320 mm² or less, more preferably 1 mm² or more and 80 mm² or less, still more preferably 1.5 mm² or more and 20 mm² or less. The term “average projected area” means a number average determined as follows: 10 solid particles that have been randomly sampled are adopted as measurement objects; and under a state in which the solid particles are mounted in the most stable state on a horizontal plane, areas projected onto the horizontal plane with light from directly above are measured, and their number average is determined.

When the solid particle has a spherical shape or a substantially spherical shape, its diameter is preferably 0.5 mm or more, more preferably 1 mm or more, still more preferably 1.5 mm or more, and is preferably 20 mm or less, more preferably 10 mm or less, still more preferably 5 mm or less. The diameter of the solid particle having a spherical shape or a substantially spherical shape is preferably 0.5 mm or more and 20 mm or less, more preferably 1 mm or more and 10 mm or less, still more preferably 1.5 mm or more and 5 mm or less.

In addition, when the solid particle has a spheroidal shape or a substantially spheroidal shape, its diameter is a circle-equivalent diameter determined from the above-mentioned horizontal projection. The diameter is preferably 0.5 mm or more, more preferably 1 mm or more, still more preferably 1.5 mm or more, and is preferably 20 mm or less, more preferably 10 mm or less, still more preferably 5 mm or less. The diameter of the solid particle having a spheroidal shape or a substantially spheroidal shape is preferably 0.5 mm or more and 20 mm or less, more preferably 1 mm or more and 10 mm or less, still more preferably 1.5 mm or more and 5 mm or less.

Further, when the solid particle has a spheroidal shape or a substantially spheroidal shape, its height is a distance between a plane that is brought into contact with the solid particle, the plane being horizontal to the horizontal plane on which the solid particle is mounted, and being positioned at a place most distant therefrom, and the horizontal plane. The height is preferably 0.4 mm or more, more preferably 0.8 mm or more, still more preferably 1.2 mm or more, and is preferably 16 mm or less, more preferably 8 mm or less, still more preferably 4 mm or less. The height of the solid particle having a spheroidal shape or a substantially spheroidal shape is preferably 0.4 mm or more and 16 mm or less, more preferably 0.8 mm or more and 8 mm or less, still more preferably 1.2 mm or more and 4 mm or less.

The average mass of the solid particles per particle is preferably 1 mg or more, more preferably 5 mg or more, still more preferably 10 mg or more. In addition, the average mass of the solid particles per particle is preferably 10,000 mg or less, more preferably 5,000 mg or less, still more preferably 1,000 mg or less. The average mass of the solid particles is preferably 1 mg or more and 10,000 mg or less, more preferably 5 mg or more and 5,000 mg or less, still more preferably 10 mg or more and 1,000 mg or less. The term “average mass” means the number average of the masses of 10 solid particles that have been randomly sampled.

The average strength of the solid particles per particle is preferably 0.14 N or more, more preferably 0.16 N or more from the viewpoint of transportation resistance. In addition, the average strength of the solid particles per particle is preferably 3 N or less, more preferably 1 N or less from the viewpoint of a sense of use. The average strength of the solid particles is preferably 0.14 N or more and 3 N or less, more preferably 0.16 N or more and 1 N or less. The term “average strength” means the number average of the strengths of 10 solid particles that have been randomly sampled. The strength of the solid particle is measured by a method described in Examples.

[Method of producing Solid Particle]

A method of producing a solid particle according to at least one embodiment of the present invention includes the following:
heating a raw material composition containing an oily component to impart fluidity thereto;
granulating the raw material composition having imparted thereto the fluidity to provide a granular raw material; and
bringing the granular raw material and powder into contact with each other under a state in which vibration is applied to the powder to coat the surface of the granular raw material with the powder.

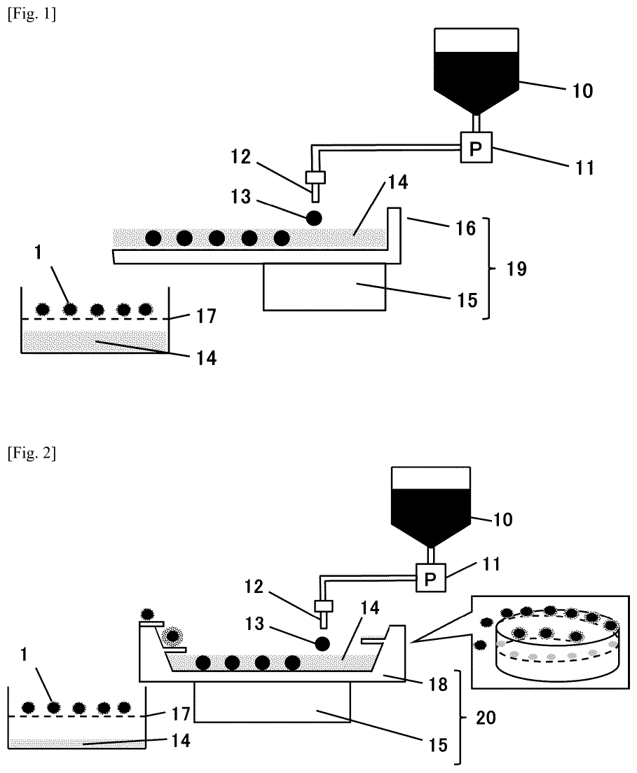

An example of the method of producing a solid particle according to at least one embodiment of the present invention is described below with reference to FIG. 1. The step 1 is heating a raw material composition to provide a raw material composition 10 having imparted thereto fluidity. In addition, the step 2 is granulating the raw material composition 10 having imparted thereto the fluidity to provide a granular raw material 13, and in FIG. 1, the granular raw material 13 is obtained by: delivering the raw material composition 10 having imparted thereto the fluidity with a pump 11; and ejecting the composition from the tip of a nozzle 12 to granulate the composition. Further, the step 3 is bringing the granular raw material 13 and powder 14 into contact with each other under a state in which vibration is applied to the powder 14 to cause the powder 14 to adhere to the granular raw material 13, to thereby coat the surface of the granular raw material 13 with the powder 14. In FIG. 1, the use of a vibration feeder 19 in which a trough 16 is arranged on a vibration device 15 applies the vibration to the powder 14 on the trough 16. It is preferred that a solid particle 1 produced through the steps 1 to 3 be conveyed on the trough 16 and separated from the powder 14 by a sieve 17.

In the production method according to at least one embodiment of the present invention, the phrase “the powder 14 adheres to the granular raw material 13” means not only that the powder 14 adheres to the surface of the granular raw material 13 but also that part of the powder 14 is incorporated into the inside of the granular raw material 13 near its surface. For example, the powder 14 is incorporated to a depth of about 100 μm from the surface of the granular raw material 13.

In addition, in the production method according to at least one embodiment of the present invention, the expression “coat the surface of the granular raw material 13 with the powder 14” means not only that the entirety of the surface of the granular raw material 13 is coated with the powder 14 but also that at least part of the surface of the granular raw material 13 is coated with the powder 14. From the viewpoint of improving the adhesion resistance and transportation resistance of the solid particle, the entirety of the surface of the granular raw material 13 is preferably coated with the powder 14.

In addition, as illustrated in FIG. 2, a bowl 18 may be used instead of the trough 16 as a container that stores the powder 14. The use of a bowl feeder 20 in which the bowl 18 is arranged on the vibration device 15 applies the vibration to the powder 14 in the bowl 18. The granular raw material 13 that has been brought into contact with the powder 14 having applied thereto the vibration ascends a slope arranged on the inner wall of the bowl 18 through the vibration while its surface is coated with the powder 14. To lengthen the time period for which the granular raw material 13 and the powder 14 are brought into contact with each other, when the trough 16 is used, the trough needs to be lengthened. However, when the bowl 18 is used, space efficiency is satisfactory because an increase in winding number of its spiral in its height direction suffices for the purpose.

### [Step 1]

The step 1 is heating the raw material composition containing the oily component to impart the fluidity thereto. The raw material composition 10 having imparted thereto the fluidity can be obtained by heating the raw material composition to a temperature equal to or more than the melting point of at least one substance in the raw material composition. In addition, the raw material composition is preferably heated to its melting point or more.

A temperature for imparting the fluidity to the raw material composition is preferably 60° C. or more, more preferably 70° C. or more, still more preferably 80° C. or more, still more preferably 85° C. or more from the viewpoint of retarding the solidification of the granular raw material 13 to accelerate the adhesion of the powder 14 in the step 3. In addition, the temperature is preferably 150° C. or less, more preferably 130° C. or less, still more preferably 120° C. or less, still more preferably 115° C. or less from the viewpoint of preventing the deterioration of the raw material composition due to heat. In particular, the temperature for imparting the fluidity to the raw material composition is preferably 60° C. or more and 150° C. or less, more preferably 70° C. or more and 130° C. or less, still more preferably 80° C. or more and 120° C. or less, still more preferably 85° C. or more and 115° C. or less.

In addition, the raw material composition 10 having imparted thereto the fluidity may be in a paste state (semisolid) in which the heated raw material composition is not completely melted. The raw material composition in a paste state is preferred because the composition has some degree of viscosity, and hence can be formed into a desired shape when the granular raw material is obtained in the step 2 serving as the next step.

### [Step 2]

The step 2 is granulating the raw material composition 10 having imparted thereto the fluidity to provide the granular raw material 13. When the raw material composition 10 having imparted thereto the fluidity illustrated in FIG. 1 is a liquid obtained by heating the raw material composition to its melting point or more, the ejection of the raw material composition delivered by the pump 11 from the tip of the nozzle 12 can provide the granular raw material 13 as a droplet. In addition, when the raw material composition 10 having imparted thereto the fluidity is in a paste state, its extrusion from the tip of the nozzle 12 by the pump 11 can provide the granular raw material 13 having the sectional shape of the tip of the nozzle 12. In addition, an apparatus that turns the raw material composition 10 having imparted thereto the fluidity into the granular raw material 13 is not limited to the apparatus illustrated in FIG. 1, and a known droplet-producing apparatus, extrusion granulator, or the like may be used.

The size of the granular raw material 13 obtained in the step 2 is substantially the same as the total size of: the core portion of the solid particle serving as the object of the production method according to at least one embodiment of the present invention; and the shell portion thereof formed of a layer of the raw material composition having incorporated thereinto the powder, the shell portion coating the core portion. In other words, in the granular raw material 13, the size of the solid particle 1 to be obtained is preferably adjusted to a range, a diameter, and/or a weight represented by the average projected area at the time of its mounting on the plane described above.

To this end, for example, when the granular raw material 13 is obtained as a droplet by ejecting the raw material composition 10 that is a liquid from the tip of the nozzle 12, the inner diameter of the nozzle 12 may be changed in accordance with the particle diameter of the target solid particle. The inner diameter of the nozzle 12 is preferably 0.5 mm or more, more preferably 1 mm or more, still more preferably 1.5 mm or more from the viewpoint of obtaining a particle diameter corresponding to the usage amount of the solid particle 1 per time. In addition, the inner diameter is preferably 20 mm or less, more preferably 10 mm or less, still more preferably 5 mm or less from the viewpoint of stably dropping the granular raw material 13. In particular, the inner diameter of the nozzle 12 is preferably 0.5 mm or more and 20 mm or less, more preferably 1 mm or more and 10 mm or less, still more preferably 1.5 mm or more and 5 mm or less.

In addition, when the raw material composition 10 having imparted thereto the fluidity is in a paste state, the granular raw material 13 corresponding to the size and weight of the target solid particle can be obtained by cutting the raw material composition 10 extruded from the tip of the nozzle 12 so that the composition may have a predetermined size.

### [Step 3]

The step 3 is bringing the granular raw material 13 and the powder 14 into contact with each other to coat the surface of the granular raw material 13 with the powder 14. In addition, the granular raw material 13 and the powder 14 are brought into contact with each other under a state in which the vibration is applied to the powder 14. When the granular raw material 13 and the powder 14 are brought into contact with each other under a state in which the vibration is applied to the powder 14, it is conceivable that the powder 14 can be incorporated to a depth of at least about 80 μm from the surface of the granular raw material 13, and hence a solid particle excellent in adhesion resistance and transportation resistance is obtained.

As illustrated in FIG. 1, the granular raw material 13 and the powder 14 are preferably brought into contact with each other by dropping the granular raw material 13 onto the powder 14 on the trough 16 arranged on the vibration device 15 or causing the raw material 13 to fall onto the powder 14. In addition, it is preferred that the powder 14 be continuously supplied onto the trough 16 by a powder-supplying device (not shown).

The distance by which the granular raw material 13 is dropped or caused to fall, that is, a distance between the tip of the nozzle 12 and the outermost surface of the layer of the powder 14 is preferably 35 cm or less, more preferably 25 cm or less, still more preferably 15 cm or less from the viewpoints of: alleviating impact when the granular raw material 13 that has been dropped or caused to fall is brought into contact with the powder 14 to prevent the deformation of the raw material; and suppressing the cooling of the granular raw material 13 during its drop or fall.

The thickness of the layer of the powder 14 is preferably 3 mm or more, more preferably 5 mm or more, still more preferably 8 mm or more from the viewpoint of accelerating the adhesion of the powder 14 to the upper portion of the granular raw material 13 that has been dropped or caused to fall. In addition, the thickness of the layer of the powder 14 is preferably 50 mm or less, more preferably 30 mm or less, still more preferably 20 mm or less, still more preferably 15 mm or less from the viewpoint of avoiding excessive use of the powder.

The amplitude of the vibration to be applied to the powder 14 when the powder 14 is brought into contact with the surface of the granular raw material 13 is preferably 0.3 mm or more, more preferably 0.4 mm or more, still more preferably 0.5 mm or more, still more preferably 0.6 mm or more, still more preferably 0.8 mm or more from the viewpoint of accelerating the adhesion of the powder 14 on the surface of the granular raw material 13. In addition, the amplitude of the vibration is preferably 5 mm or less, more preferably 4 mm or less, still more preferably 3 mm or less, still further more preferably 1.5 mm or less, still further more preferably 1.4 mm or less, still further more preferably 1.3 mm or less. In particular, the amplitude of the vibration is preferably 0.3 mm or more and 5 mm or less, more preferably 0.4 mm or more and 4 mm or less, still more preferably 0.5 mm or more and 4 mm or less, still more preferably 0.5 mm or more and 3 mm or less, still further more preferably 0.6 mm or more and 3 mm or less, still further more preferably 0.8 mm or more and 3 mm or less, still further more preferably 0.3 mm or more and 1.5 mm or less, still further more preferably 0.4 mm or more and 1.4 mm or less, still further more preferably 0.5 mm or more and 1.3 mm or less, still further more preferably 0.6 mm or more and 1.3 mm or less.

The amplitude of the vibration to be applied to the powder 14 is preferably measured at a position directly above the vibration device 15.

In addition, the frequency of the vibration is preferably 30 Hz or more, more preferably 40 Hz or more, still more preferably 50 Hz or more from the viewpoint of accelerating the adhesion of the powder 14 to the surface of the granular raw material 13. In addition, the frequency of the vibration is preferably 300 Hz or less, more preferably 100 Hz or less, still more preferably 75 Hz or less, still further more preferably 60 Hz or less. In particular, the frequency of the vibration is preferably 30 Hz or more and 300 Hz or less, more preferably 40 Hz or more and 100 Hz or less, still more preferably 50 Hz or more and 75 Hz or less, still further more preferably 50 Hz or more and 60 Hz or less.

The temperature of the powder 14 is preferably 5° C. or more, more preferably 15° C. or more, still more preferably 20° C. or more from the viewpoint of retarding the solidification of the granular raw material 13 to accelerate the adhesion of the powder 14. In addition, he temperature of the powder 14 is preferably 60° C. or less, more preferably 55° C. or less, still more preferably 50° C. or less from the viewpoint of suppressing excessive adhesion of the powder 14 to the granular raw material 13. In particular, the temperature of the powder 14 is preferably 5° C. or more and 60° C. or less, more preferably 15° C. or more and 55° C. or less, still more preferably 20° C. or more and 50° C. or less. In addition, the temperature of the powder 14 when the powder 14 is brought into contact with the surface of the granular raw material 13 is preferably normal temperature (from 20° C. to 30° C.).

The time period for which the granular raw material 13 and the powder 14 are brought into contact with each other is preferably 2 seconds or more, more preferably 3 seconds or more, still more preferably 4 seconds or more from the viewpoint of accelerating the adhesion of the powder on the surface of the granular raw material 13. In addition, the time period for which the granular raw material 13 and the powder 14 are brought into contact with each other is preferably 30 seconds or less, more preferably 15 seconds or less, still more preferably 10 seconds or less from the viewpoint of productivity. In particular, the time period for which the granular raw material 13 and the powder 14 are brought into contact with each other is preferably 2 seconds or more and 30 seconds or less, more preferably 3 seconds or more and 15 seconds or less, still more preferably 4 seconds or more and 10 seconds or less.

Through the foregoing step 3, the powder 14 is caused to adhere to the surface of the granular raw material 13, and hence the surface of the granular raw material 13 is coated with the powder 14. Thus, the solid particle 1 can be obtained. The production method may include cooling the granular raw material 13 as an additional step simultaneously with the step 3 and/or after the step 3. The performance of the cooling can further secure the adhesion of the powder 14 to the granular raw material 13. The cooling may be, for example, natural cooling, or may be forced cooling.

When the natural cooling is performed, the solid particle 1 in which the surface of the granular raw material 13 is coated with the powder 14 only needs to be left at rest under room temperature.

When the forced cooling is performed, the following only needs to be performed: a gas is blown onto the solid particle 1; the solid particle 1 is left at rest in a refrigerator; or the solid particle 1 is brought into contact with a refrigerant.

The granular raw material 13 softens at the time point when the powder 14 is brought into contact with the surface of the granular raw material 13. The granular raw material 13 whose surface has been coated with the powder 14 is solidified at any subsequent stage. The term “solidification” means that cooling makes the hardness of the granular raw material 13 equal to the hardness of the raw material composition before the fluidity is imparted thereto.

A portion free of the powder 14 in which the granular raw material 13 is solidified is the core portion of the solid particle 1. The granular raw material 13 is obtained by granulating the raw material composition, and is in a state before its solidification. In addition, a portion where the raw material composition incorporates the powder 14 in the inside of the granular raw material 13 near its surface is the shell portion of the solid particle 1.

After the step 3, the powder 14 that does not stick to the surface of the solid particle 1 may be removed prior to the cooling of the solid particle 1, after the cooling, or simultaneously with the cooling. The term “powder 14 that does not stick to the surface of the solid particle 1” refers to the powder 14, which adheres to the surface to such a weak extent as to fall from the solid particle 1 owing to an external force such as vibration to be applied at the time of, for example, the conveyance of the solid particle 1, or which does not adhere to any portion derived from the raw material composition at all.

The sieve 17 having such an aperture as not to pass the solid particle 1 therethrough as illustrated in FIG. 1 may be used in the removal of the powder.

The thickness of the shell portion of the solid particle 1 after the removal of the powder is preferably 80 μm or more, more preferably 100 μm or more, still more preferably 110 μm or more, still further more preferably 120 μm or more, still further more preferably 130 μm or more, still further more preferably 150 μm or more from the viewpoint of improving the adhesion resistance and transportation resistance thereof. In particular, the thickness of the shell portion is preferably 110 μm or more because the adhesion resistance and transportation resistance of the solid particle 1 are further improved. In addition, the thickness is preferably 300 μm or less, more preferably 250 μm or less, still more preferably 200 μm or less from the viewpoint of suppressing a reduction in sense of use of the particle.

The ratio of the powder incorporated into the solid particle 1, after the removal of the powder that is free from adhering to the solid particle, to the mass of the solid particle is preferably 5.0 mass % or more, more preferably 7.0 mass % or more, still more preferably 7.5 mass % or more, still further more preferably 8.0 mass % or more from the viewpoint of improving the adhesion resistance and transportation resistance of the particle. In addition, the ratio is preferably 30 mass % or less, more preferably 20 mass % or less, still more preferably 15 mass % or less, still further more preferably 13 mass % or less, still further more preferably 12 mass % or less from the viewpoint of eliminating an influence on the sense of use thereof.

The solid particle produced by the production method according to at least one embodiment of the present invention has adhesion resistance because the surface of the core portion is coated with the shell portion containing the powder. Accordingly, for example, even when the core portion has stickiness, the stickiness is lessened by the shell portion containing the powder, and hence the following effect is exhibited: unintended adhesion of the solid particle to a conveying device at the time of the conveyance of the solid particle is prevented. In addition, when the solid particle is filled into, for example, a container, the following effect is exhibited: a reduction in fillability due to unintended adhesion of the solid particle to the inner wall or lid of the container, and a reduction in property by which the solid particle is removed from the container owing to unintended adhesion of the solid particle to the inner wall of the container or any other solid particle after its filling into the container are prevented. Meanwhile, the strength of the solid particle itself is improved by not only the adhesion of the powder to the surface of the solid particle but also the incorporation of the powder into the inside of the solid particle near its surface, and hence the resistance thereof to impact involved in its transportation or the like is improved. Further, the powder is localized to the shell portion near the surface of the solid particle. Accordingly, the substantial content of the powder in the solid particle is suppressed, and hence a satisfactory sense of use is exhibited. Accordingly, the solid particle produced by the production method according to at least one embodiment of the present invention has adhesion resistance, and can achieve both of transportation resistance and a sense of use.

The solid particle produced by the production method according to at least one embodiment of the present invention is preferably formed into at least one kind of shape selected from a spherical shape, a substantially spherical shape, a spheroidal shape, and a substantially spheroidal shape in view of the characteristics of the step 2 and the step 3. That is, the droplet obtained in the step 2 by the ejection of the raw material composition 10 having imparted thereto the fluidity from the tip of the nozzle 12 has a spherical shape or a substantially spherical shape by virtue of the surface tension of the raw material composition 10. Then, in the step 3, the granular raw material 13 may be deformed into a spheroidal shape or a substantially spheroidal shape when dropped onto the powder 14, but is suppressed from being further deformed because the only external force to be applied thereafter is the vibration applied to the powder 14. When the surface of the granular raw material is coated with the powder by a method except the application of the vibration, for example, when a particle corresponding to the solid particle according to at least one embodiment of the present invention is produced by stirring the granular raw material in the powder, an increase in thickness of the shell portion requires an increase in stirring strength, but the increase in stirring strength may heavily deform the solid particle. Meanwhile, according to at least one embodiment of the present invention, the thickness of the shell portion can be increased while the deformation of the solid particle is suppressed.

In addition, the ratio of the height of the solid particle produced by the production method according to at least one embodiment of the present invention to the diameter thereof is preferably 40% or more, more preferably 45% or more, still more preferably 50% or more from the viewpoint of the satisfactoriness of the appearance thereof.

### [Raw Material for Solid Particle]

### [Raw Material Composition]

The raw material composition is a solid at room temperature (25° C.), and has a melting point of preferably 50° C. or more, more preferably 55° C. or more, still more preferably 60° C. or more. When the raw material composition has a melting point equal to or more than those temperatures, the sense of use of the solid particle at the time of its use as a cosmetic can be improved because the granular raw material obtained by granulating the raw material composition serves as the core portion of the solid particle. In addition, the melting point of the raw material composition is preferably 150° C. or less, more preferably 120° C. or less, still more preferably 110° C. or less from the viewpoint of ease of production. Also when the raw material composition has a melting point equal to or less than those temperatures, the sense of use of the solid particle can be improved. In particular, the melting point of the raw material composition is preferably 50° C. or more and 150° C. or less, more preferably 55° C. or more and 120° C. or less, still more preferably 60° C. or more and 110° C. or less.

The raw material composition typically contains a plurality of substances. In this case, the melting point of the raw material composition is measured by any one of the first method, second method, and third method of the general test methods of the Japanese Standards of Quasi-drug Ingredients. Which one of the methods is adopted is selected mainly by the melting point of the raw material composition. When the melting point is as high as more than 75° C., the first method may be used, when the melting point is 50° C. or more and 75° C. or less, the second method may be used, and when the melting point is less than 50° C., the third method may be used.

The raw material composition is preferably a cosmetic raw material composition from the viewpoint that the solid particle according to at least one embodiment of the present invention is preferably used as a cosmetic.

The raw material composition preferably has a continuous phase formed of one or two or more kinds of oily components. In some cases, the raw material composition contains a powder component such as a pigment dispersed in the continuous phase.

Examples of the oily component include a hydrocarbon oil, an ester oil, an ether oil, a fatty acid, an alcohol, a silicone oil, and a fluorine oil. The number of carbons in the oily component is preferably 6 or more, more preferably 10 or more, and is preferably 50 or less, more preferably 30 or less. Specific examples of the oily component include: waxes including: mineral waxes, such as ozokerite and ceresin; petroleum waxes, such as a paraffin and a microcrystalline wax; synthetic hydrocarbons, such as a Fischer-Tropsch wax, a polyethylene wax, and a synthetic wax; plant waxes, such as a carnauba wax, a candelilla wax, a rice wax, a sunflower wax, a hydrogenated jojoba oil, and a Japanese wax; animal waxes, such as a beeswax and a whale wax; and synthetic waxes, such as a silicone wax, a synthetic beeswax, and a synthetic Japanese wax; oily gelling agents, such as dextrin palmitate, a sucrose fatty acid ester, inulin stearate, 12-hydroxystearic acid, dibutyl lauroyl glutamide, dibutyl ethylhexanoyl glutamide, and a polyamide resin; paste oils, such as vaseline, a vinyl leather wax, dipentaerythrityl hexa (behenate/benzoate/ethylhexanoate), cholesteryl hydroxystearate, dipentaerythrityl tetra(hydroxystearate/isostearate), hydrogenated palm oil, dipentaerythrityl hexahydroxystearate, glyceryl tri (caprylate/caprate/myristate/stearate), dipentaerythrityl hexa (hydroxystearate/stearate/rosinate), phytosteryl oleate, glyceryl (ethylhexanoate/stearate/adipate), di (octyldodecyl/phytosteryl/behenyl) lauroyl glutamate, (phytosteryl/isostearyl/cetyl/stearyl/behenyl)dimer dilinoleate, dimer dilinoleyl bis(behenyl/isostearyl/phytosteryl)dimer dilinoleate, hard lanolin, reduced lanolin, and bis-diglyceryl polyacyladipate-2; linear or branched hydrocarbon oils, such as a liquid paraffin, a light liquid isoparaffin, a heavy liquid isoparaffin, a mineral oil, squalane, an α-olefin oligomer, polyisobutylene, polybutene, hydrogenated polyisobutene, and hydrogenated polydecene; ester oils, such as isononyl isononanoate, isodecyl isononanoate, isotridecyl isononanoate, tricyclodecanemethyl isononanoate, ethyl isostearate, isobutyl isostearate, isopropyl isostearate, 2-hexyldecyl isostearate, di-2-ethylhexyl succinate, bis-ethoxydiglycol succinate, hexyl laurate, propanediol di(caprylate/caprate), neopentyl glycol diisononanoate, neopentyl glycol dicaprate, glyceryl diisostearate, polyglyceryl diisostearate, propanediol diisostearate, trimethylolpropane triisostearate, glyceryl triisostearate, diglyceryl triisostearate, diglyceryl tetraisostearate, diisostearyl malate, octyldodecyl malate, a glycerin fatty acid ester, jojoba oil, di(phytosteryl/octyldodecyl) lauroyl glutamate, octyldodecyl myristate, isopropyl myristate, 2-ethylhexyl palmitate, isopropyl palmitate, cetyl 2-ethylhexanoate, trimethylolpropane tri-2-ethylhexanoate, glyceryl tri-2-ethylhexanoate, octyldodecyl myristate, 2-hexyldecyl myristate, 2-hexyldecyl 2-ethylhexanoate, neopentyl glycol di-2-ethylhexanoate, ethylhexyl hydroxystearate, glyceryl tri (caprylate/caprate), glyceryl trioctanoate, neopentyl glycol dioctanoate, tridecyl trimellitate, dipentaerythrityl tetraisostearate, pentaerythrityl tetraisostearate, octyl methoxycinnamate, 2-ethylhexyl paramethoxycinnamate, diisopropyl dimerate, and propylene carbonate; higher alcohols, such as lauryl alcohol, oleyl alcohol, isostearyl alcohol, behenyl alcohol, and octyldodecanol; silicone oils, such as dimethylpolysiloxane, dimethylcyclopolysiloxane, methylphenylpolysiloxane, trimethylpentaphenyltrisiloxane, methylhydrogenpolysiloxane, higher alcohol-modified organopolysiloxane, and bisalkyl(C16-18)glycerin undecyl dimethicone; fluorine oils, such as a fluoropolyether, a perfluoroalkyl ether silicone, and a fluorine-modified silicone; and tocopherol, dipropylene glycol, and phenoxyethanol. Those oily components may be used alone or in combination thereof.

The raw material composition contains preferably 40 mass % or more, more preferably 50 mass % or more, still more preferably 60 mass % or more, still more preferably 70 mass % or more, still more preferably 80 mass % or more of the oily component in terms of the sense of use of the solid particle. From the same viewpoint, the raw material composition contains preferably 100 mass % or less, more preferably 99 mass % or less, still more preferably 98 mass % or less, still more preferably 97 mass % or less, still more preferably 95 mass % or less of the oily component. In particular, the raw material composition contains preferably 40 mass % or more and 100 mass % or less, more preferably 50 mass % or more and 99 mass % or less, still more preferably 60 mass % or more and 98 mass % or less, still more preferably 70 mass % or more and 97 mass % or less, still more preferably 80 mass % or more and 95 mass % or less of the oily component.

The raw material composition preferably contains an oily component that is in a solid state at 20° C. (has a melting point of more than 20° C.) (e.g., any one of the above-mentioned waxes) from the viewpoint of improving the shape-retaining property of the solid particle. The raw material composition contains preferably 3 mass % or more, more preferably 5 mass % or more of the oily component that is in a solid state at 20° C. from the viewpoint of the shape-retaining property of the solid particle. In addition, the raw material composition contains preferably 30 mass % or less, more preferably 10 mass % or less of the oily component that is in a solid state at 20° C. from the viewpoint of the sense of use of the solid particle. In particular, the raw material composition contains preferably 3 mass % or more and 30 mass % or less, more preferably 5 mass % or more and 10 mass % or less of the oily component that is in a solid state at 20° C. In addition, the raw material composition preferably contains an oily component that is in a liquid state at 20° C. (has a melting point of 20° C. or less) from the viewpoint of improving the sense of use of the solid particle. From this viewpoint, the raw material composition contains preferably 18 mass % or more, more preferably 25 mass % or more, still more preferably 35 mass % or more of the oily component that is in a liquid state at 20° C. From the same viewpoint, the raw material composition contains preferably 97 mass % or less, more preferably 95 mass % or less, still more preferably 93 mass % or less of the oily component that is in a liquid state at 20° C. In particular, the raw material composition contains preferably 18 mass % or more and 97 mass % or less, more preferably 25 mass % or more and 95 mass % or less, still more preferably 35 mass % or more and 93 mass % or less of the oily component that is in a liquid state at 20° C.

Whether or not the oily component is in a liquid state at 20° C. can be judged by contents described in the section “Physical State” of the safety data sheet (SDS) of each component. When the oily component that is in a liquid state at 20° C. is incorporated in the above-mentioned ranges into the raw material composition, the oily component is present on the surface of the core portion of the solid particle after its production, and the core portion is liable to be bonded to any other thing owing to the fact. In contrast, in the solid particle produced by the production method according to at least one embodiment of the present invention, the surface of the core portion is coated with the shell portion. Accordingly, unintended bonding between the solid particles or between the particle and any other thing is suppressed, and hence the particle has adhesion resistance.

For example, various components that have heretofore been used in cosmetics may each be used as the powder component in the raw material composition without any particular limitation. The powder component may be inorganic powder, or may be organic powder. The inorganic powder and the organic powder may be used in combination. The shape of a particle for forming the powder component is not particularly limited, and may be, for example, a spherical shape, a polyhedral shape, a flake shape, a spindle shape, a fibrous shape, an indefinite shape, or a combination thereof.

Pigments, such as a coloring pigment, a luster pigment, and an extender pigment, may each be used as the powder component in the raw material composition, and an inorganic powder pigment is preferred.

Examples of the coloring pigment include: metal oxides, such as titanium oxide, zinc oxide, yellow iron oxide, red iron oxide, black iron oxide, iron blue, ultramarine blue, chromium oxide, and chromium hydroxide; metal complexes, such as manganese violet and cobalt titanate; inorganic pigments such as carbon black; synthetic organic pigments, such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 405, Red No. 505, Orange No. 203, Orange No. 204, Orange No. 205, Yellow No. 4, Yellow No. 5, Yellow No. 401, Blue No. 1, and Blue No. 404; and natural organic colors, such as β-carotene, caramel, and a paprika color.

Examples of the luster pigment include: a pigment obtained by coating the surface of sheet-shaped powder of mica, synthetic fluorophlogopite, glass, silica, alumina, or talc with a colorant including titanium oxide, iron oxide, silicon oxide, iron blue, chromium oxide, tin oxide, chromium hydroxide, gold, silver, carmine, or an organic pigment, such as Red No. 202 or Yellow No. 4; and a pigment obtained by cutting a raw material film of polyethylene terephthalate/polymethyl methacrylate laminated powder, polyethylene terephthalate/aluminum deposited powder, or polyethylene terephthalate/gold deposition-laminated powder into any appropriate shape.

Examples of the extender pigment include inorganic powders including silica, mica, synthetic fluorophlogopite, glass powder, barium sulfate, kaolin, bentonite, hectorite, zeolite, bismuth oxychloride, zirconium oxide, magnesium oxide, aluminum oxide, calcium sulfate, barium sulfate, magnesium sulfate, calcium carbonate, magnesium carbonate, and talc.

Further examples thereof include organic powders including nylon, polyethylene, a silicone elastomer such as a (vinyl dimethicone/methicone silsesquioxane) crosspolymer, polymethyl methacrylate, lauroyl lysine, silk powder, cellulose powder, a dispersant such as a polyvalent metal salt of a long-chain fatty acid, and various wax powders.

The ratio of the powder component in the raw material composition is preferably 0.01 mass % or more, more preferably 0.1 mass % or more, still more preferably 1 mass % or more, still more preferably 3 mass % or more, still more preferably 5 mass % or more. In addition, the ratio of the powder component in the raw material composition is preferably 60 mass % or less, more preferably 50 mass % or less, still more preferably 45 mass % or less, still more preferably 30 mass % or less, still more preferably 20 mass % or less. In particular, the ratio of the powder component in the raw material composition is preferably 0.01 mass % or more and 60 mass % or less, more preferably 0.1 mass % or more and 50 mass % or less, still more preferably 1 mass % or more and 45 mass % or less, still more preferably 3 mass % or more and 30 mass % or less, still more preferably 5 mass % or more and 25 mass % or less.

The hardness of the core portion is one factor that affects, for example, the sense of use of the solid particle. From this viewpoint, the hardness of the raw material composition is preferably 500 g or less, more preferably 350 g or less, still more preferably 250 g or less, still further more preferably 150 g or less. Meanwhile, unintended bonding between the raw material composition and any other thing can be suppressed by setting the hardness of the raw material composition to preferably 0.5 g or more, more preferably 5 g or more, still more preferably 15 g or more. From those viewpoints, the hardness of the raw material composition is preferably 0.5 g or more and 500 g or less, more preferably 5 g or more and 350 g or less, still more preferably 15 g or more and 250 g or less, still further more preferably 15 g or more and 150 g or less. The hardness of the raw material composition is measured by a method described in Examples.

### [Powder]

The ratio (powder adhesion ratio) of the powder to be used in the step 3 to the mass of the solid particle is preferably 5 mass % or more, more preferably 7.0 mass % or more, still more preferably 7.5 mass % or more, still further more preferably 8.0 mass % or more from the viewpoints of the adhesion resistance and transportation resistance of the particle. In addition, the ratio is preferably 30 mass % or less, more preferably 20 mass % or less, still more preferably 15 mass % or less, still further more preferably 13 mass % or less, still further more preferably 12 mass % or less from the viewpoint of eliminating an influence on the sense of use thereof.

The same powder as that generally used in a cosmetic may be used as the powder without any particular limitation. The powder may be inorganic powder, or may be organic powder. Although the inorganic powder and the organic powder may be used in combination, the inorganic powder is preferably incorporated, an inorganic powder pigment is more preferred, and silica is still more preferred.

The raw material composition contains the oily component. Accordingly, a case in which inorganic powder having high hydrophilicity such as silica is used as the powder is preferred because the contact angle of the raw material composition with respect to the powder increases, and hence the shape of the solid particle to be obtained easily becomes at least one kind of shape selected from a spherical shape, a substantially spherical shape, a spheroidal shape, and a substantially spheroidal shape.

Specifically, the same component as the powder component in the raw material composition described above may be used.

Powder having such a size that the adhesion of the solid particle serving as the object of the production method according to at least one embodiment of the present invention to any other thing in its production process is effectively prevented is suitably used as the powder. For example, when the sizes of particles for forming the powder are represented by a volume cumulative particle diameter D₅₀ at a cumulative volume of 50 vol % measured by a laser diffraction/scattering particle size distribution measurement method, the D₅₀ is preferably 0.01 μm or more, more preferably 0.1 μm or more, still more preferably 1 μm or more, still more preferably 5 μm or more, still more preferably 10 μm or more. The use of the powder having such size facilitates the performance of the operation of removing the powder. In addition, the D₅₀ is preferably 500 μm or less, more preferably 300 μm or less, still more preferably 160 μm or less, still more preferably 100 μm or less, still more preferably 30 μm or less. The use of the powder having such size can cause the powder to be easily to adhere to the surface of the granular raw material. In particular, the D₅₀ is preferably 0.01 μm or more and 500 μm or less, more preferably 0.1 μm or more and 300 μm or less, still more preferably 1 μm or more and 160 μm or less, still more preferably 5 μm or more and 100 μm or less, still more preferably 10 μm or more and 30 μm or less.

The oil absorption of the powder is preferably 5 mL/100 g or more, more preferably 15 mL/100 g or more, still more preferably 20 mL/100 g or more from the viewpoint of effectively preventing the adhesion of the solid particle to any other thing in its production process. In addition, the oil absorption of the powder is preferably 500 mL/100 g or less, more preferably 400 mL/100 g or less, still more preferably 350 mL/100 g or less from the viewpoint of preventing the powder from excessively absorbing the oily component in the granular raw material. The oil absorption is measured in conformity with JIS K 5101-13-1:2004.

The amount of the powder in the solid particle may be determined and measured by various methods. For example, when the powder is inorganic matter, its amount may be determined by burning organic matter in the solid particle and measuring its remaining ratio with a thermogravimetric differential thermal analyzer as described below. The amount of the powder that is inorganic matter is measured by a method described in Examples.

When the powder is organic matter, the amount of the powder adhering to the core portion may be determined as follows: a product obtained by dissolving all the components of the solid particle in a solvent is used as a sample; and the sample is subjected to measurement, such as ¹H-NMR or laser desorption/ionization mass spectrometry (LDI-MS).

The solid particle produced by the production method according to at least one embodiment of the present invention is preferably used as a cosmetic. The solid particle may be used in, for example, a makeup method including collapsing the particle and applying the collapsed particle to a human body for cosmetic purposes. Specifically, the following may be performed: the solid particle is mounted on a cosmetic palette, and is collapsed with a makeup brush, followed by its application to a lip with the makeup brush like a lipstick. Alternatively, the following may be performed: the solid particle is collapsed on the back of a hand, and is applied to a cheek with a finger like a blusher or a concealer. Alternatively, the following may be performed: the solid particle is collapsed on the back of a hand, and is applied to a finger or the back of the hand with the finger like a hand cream. Further, the solid particle may be used like an oil cleansing by being collapsed with a hand. Further, the solid particle may be used like a treatment or a hair wax by being collapsed with a hand or a tool and applied to hair.

The present invention has been described above on the basis of its exemplary embodiments. However, the present invention is not limited to the embodiments.

### EXAMPLES

The present invention is described in more detail below by way of Examples.

However, the scope of the present invention is not limited to these Examples.

### [Preparation of Raw Material Composition]

The composition of a raw material composition for a lipstick is shown in Table 1 below, and the composition of a raw material composition for an eye shadow is shown in Table 2 below. In each of the raw material compositions, base raw materials (raw material composition excluding a coloring pigment and an extender pigment) were heated and dissolved at 110° C. for 30 minutes, and were uniformly mixed with a disper. Next, the coloring pigment and the extender pigment were added to the base raw materials, and the materials were further uniformly mixed for 15 minutes, followed by degassing. After that, the mixture was naturally cooled to be solidified. Thus, the raw material compositions were prepared. The melting points of the prepared raw material compositions were measured in accordance with the general test methods of the Japanese Standards of Quasi-drug Ingredients. As a result, the raw material composition for a lipstick had a melting point of 70° C., and the raw material composition for an eye shadow had a melting point of 79° C.

The raw material composition for a lipstick prepared in the foregoing was heated and dissolved at 115° C., and was filled into a resin-made ointment jar (having a diameter of 30 mm and a height of 14 mm) up to a height of 10 mm. After that, the composition was cooled at 20° C. for 2 hours to be solidified, and was left at rest at 30° C. for 6 hours or more. After that, the hardness of the raw material composition for a lipstick was measured with a rheometer manufactured by Rheotech by reading the maximum of a load when a jig having a diameter of 2 mm was caused to penetrate into the composition to a depth of 2 mm at a table speed of 2 mm/s. As a result, the hardness was 35 g.

The hardness of the raw material composition for an eye shadow prepared in the foregoing was measured under the same conditions. As a result, the hardness was 41 g.

TABLE 1

| | | Ratio (mass %) |
|---|---|---|
| Base | Paraffin | 4.0 |
| | Polyethylene wax | 2.0 |
| | Microcrystalline wax | 2.0 |
| | (Multi-branched isostearic acid) | 30.0 |
| | dipentaerythrityl tetraisostearate | |
| | Hydrogenated polyisobutene | 10.0 |
| | Di(phytosteryl/octyldodecyl) lauroyl | 10.0 |
| | glutamate | |
| | Octyldodecanol | 15.0 |
| | Bisalkyl (C16-18) glycerin undecyl | 6.0 |
| | dimethicone | |
| | Glyceryl tri(caprylate/caprate) | 10.2 |
| | Tocopherol | 0.1 |
| | Dipropylene glycol | 0.1 |
| Coloring | Red No. 202 | 0.3 |
| pigment | Yellow No. 4 Aluminum Lake | 0.2 |
| | Yellow iron oxide | 0.5 |
| | Red iron oxide | 0.1 |
| | Titanium oxide | 1.0 |
| Luster | Mica titanium | 2.0 |
| pigmen | Titanium oxide-coated glass flake | 1.5 |
| Extender | Mica | 5.0 |
| pigment | | |

| | |
|---|---|
| Total | 100.0 |

TABLE 2

| | | Ratio (mass %) |
|---|---|---|
| Base | Paraffin | 4.0 |
| | Synthetic wax | 3.0 |
| | Microcrystalline wax | 2.0 |
| | (Multi-branched isostearic acid) | 25.0 |
| | dipentaerythrityl tetraisostearate | |
| | Polyglyceryl-2 triisostearate | 5.0 |
| | Polyglyceryl-2 diisostearate | 5.0 |
| | Isotridecyl isononanoate | 15.0 |
| | Glyceryl tri(caprylate/caprate) | 20.45 |
| Coloring | Red No. 202 | 0.05 |
| pigment | | |
| Luster | Titanium oxide-coated glass flake | 20.0 |
| pigment | | |
| Dispersant | Zinc stearate | 0.5 |

| | |
|---|---|
| Total | 100.0 |

As shown in Table 1, the amount of oily components in the raw material composition for a lipstick was the amount of components shown as bases, that is, 89.4 mass %, and the amount of powder components therein was the total amount of the coloring pigments, the luster pigments, and the extender pigment, that is, 10.6 mass %. In addition, the amount of oily components each of which had a melting point of more than 20° C. (was a solid at 20° C.) in the raw material composition was the total amount of the paraffin, the polyethylene wax, and the microcrystalline wax, that is, 8 mass %, and the amount of oily components each of which had a melting point of 20° C. or less (was a liquid at 20° C. or less) therein was the total amount of the other components, that is, 81.4 mass %.

As shown in Table 2, the amount of oily components in the raw material composition for an eye shadow was the amount of components shown as bases, that is, 79.45 mass %, and the amount of powder components therein was the total amount of the coloring pigment, the luster pigment, and the dispersant, that is, 20.55 mass %. In addition, the amount of oily components each of which had a melting point of more than 20° C. (was a solid at 20° C.) in the raw material composition for an eye shadow was the total amount of the paraffin, the synthetic wax, and the microcrystalline wax, that is, 9.0 mass %, and the amount of oily components each of which had a melting point of 20° C. or less (was a liquid at 20° C. or less) therein was the total amount of the other components, that is, 70.45 mass %.

### Example 1

### Steps 1 and 2 (Production of Granular Raw Material)

The plurality of granular raw materials 13 were obtained from the raw material composition for a lipstick with the apparatus illustrated in FIG. 1. That is, the raw material composition 10 for a lipstick having imparted thereto fluidity through the melting of the raw material composition by heating to 90° C. was delivered with the pump 11, and was ejected from the tip of the nozzle 12 having an inner diameter of 2.0 mm at a dropping temperature and by a dropping distance, the temperature and the distance being shown in Table 3. Thus, the granular raw materials 13 serving as droplets were produced. The term “dropping distance” refers to a distance between the tip of the nozzle 12 and the outermost surface of the layer of the powder 14.

Step 3 (Contact between Granular Raw Material and Powder)

The granular raw materials 13 ejected in the foregoing were dropped onto the trough 16 (dropping distance: 117 mm) onto which the powder 14 at a temperature of 25° C. having applied thereto vibration having an amplitude of 0.663 mm and a frequency of 54.0 Hz by the vibration feeder 19 (SMALL ELECTROMAGNETIC FEEDER CF-2 manufactured by Sinfonia Technology Co., Ltd.) including the vibration device 15 and the trough 16 was supplied and mounted so as to have a thickness of 10 mm, and the powder 14 was caused to adhere to the surface of each of the granular raw materials 13 by bringing the granular raw materials 13 and the powder 14 into contact with each other. A contact time shown in Table 3 is a time period required for the granular raw materials 13 to which the powder 14 has adhered to reach the outlet of the trough 16 after the dropping of the granular raw materials 13. Spherical silica having an average particle diameter D50 of 15 μm and an oil absorption of 150 mL/100 g was used as the powder 14. Thus, the target solid particles 1 for a lipstick in each of which the granular raw material 13 was coated with the powder 14 by the adhesion of the powder 14 to the surface of the granular raw material 13 were obtained.

The value of the amplitude shown in Table 3 is a value obtained by measuring an amplitude directly above the vibration device 15 on the upper surface of the trough 16 with a laser displacement meter (LK-G 5000 manufactured by Keyence Corporation). The measurement was performed under the following conditions: the measurement was performed in a diffusion-reflection mode at a sampling period of 200 μs (5 kHz) and a moving average of 4.

Only in Example 1, the amplitude was visually measured with an amplitude identification plate. The amplitude identification plate was attached to the center of the side surface of the trough 16, and a scale at the point at which oblique lines intersected with each other was visually read. As a result, the value was 1 mm, which was different from that measured with the laser displacement meter. The amplitude on the trough is small directly above the vibration device, and shows a larger value as a measurement site approaches the tip of the trough distant from the vibration device. It was conceived that the read value became larger than the value measured with the above-mentioned laser displacement meter because the center of the trough having attached thereto the amplitude identification plate was a portion somewhat distant from the vibration device.

### (Removal and Cooling of Powder)

After that, the powder 14 and the solid particles 1 were separated from each other with the sieve 17 having an aperture of 2,000 μm under room temperature (25° C.) so that the powder 14 that did not adhere to the solid particles 1 was removed. The solid particles 1 were naturally cooled on the sieve for 1 minute or more, and were recovered. The average projected area, average diameter, and average height of the solid particles calculated from the 10 solid particles 1 that had been randomly sampled were 11 mm², 3.7 mm, and 2.4 mm, respectively, and the average of the ratios of the heights to the diameters in the individual particles was 64.9%.

### [Evaluation of Solid Particles 1]

### (1) Evaluation of Adhesion Resistance

0.5 Gram of the resultant solid particles 1 were filled into a glass-made screw tube No. 02 (manufactured by Maruemu Corporation, inner diameter of the opening of the container: 5.5 mm, height: 35 mm, height after capping: 40 mm) serving as a container, and the height X (cm) of the solid particles from the bottom portion of the container to the upper surface thereof was measured. After that, the container was left at rest under an environment at 50° C. for 60 minutes, and then the container was left at room temperature (25° C.) for 1 minute. After that, the container was inverted, and the height Y (cm) of the solid particles that adhered to the bottom portion of the container newly serving as an upper side and did not fall onto the cap portion of the container newly serving as a lower side was measured. The adhesion ratio (%) of the solid particles was calculated from the following equation 1 on the basis of those results. A smaller numerical value means that the adhesion resistance of the solid particles 1 is higher. In the case where all the solid particles adhere to the bottom portion of the container, when their gravitation excels their adhesive force, the value of the Y at the time of the inversion of the container may surpass the original value of the X to result in an adhesion ratio of more than 100%. In each of Comparative Examples 1 to 3 in Table 3, all the solid particles adhered to the bottom portion of the container.

$$Adhesion ratio ( \% ) = Y / X \times 100 \qquad ( Equation 1 )$$

### (2) Evaluation of Sense of Use

The resultant solid particles 1 were collapsed on a hand, and the presence or absence of roughness derived from the powder 14 was recognized. As a result, the particles were free of roughness, and hence provided a satisfactory sense of use.

### (3) Evaluation of Transportation Resistance

0.5 Gram of the resultant solid particles 1 were filled into a glass-made screw tube No. 02 (manufactured by Maruemu Corporation, inner diameter of the opening of the container: 5.5 mm, height: 35 mm, height after capping: 40 mm) serving as a container, and the height X (cm) of the solid particles from the bottom portion of the container to the upper surface thereof was measured. A resin-made cylinder having an inner diameter of 25 mm and a height of 116 mm was vertically left at rest on a non-slip mat (material: polyethylene, thickness: 5 mm), and under an environment at room temperature (25° C.), the container was caused to fall under an upright state from above (falling distance: 112 mm). After each container had been subjected to the falling operation eight times, the container was inverted, and the height Y (cm) of the solid particles that adhered to the bottom portion of the container newly serving as an upper side and did not fall onto the cap portion of the container newly serving as a lower side was measured, followed by the calculation of the “adhesion ratio” of the solid particles 1 from (Equation 1) above. A smaller numerical value means that the transportation resistance of the solid particles 1 is higher. In the evaluation of the transportation resistance, as a result of the performance of the falling operation after the filling of the solid particles into the container, there may occur a phenomenon in which the particles tend to be slightly collapsed by the impact of the falling, or a gap between the particles is clogged. Accordingly, even when all the solid particles adhere to the bottom portion of the container, the value of the Y at the time of the inversion of the container may fall short of the original value of the X to result in an adhesion ratio of less than 100%. In each of Comparative Examples 1 to 3 in Table 3, all the solid particles adhered to the bottom portion of the container.

### (4) Determination of Powder Adhesion Ratio

The amount (adhesion ratio) of the silica used as the powder 14 in the solid particles 1 was determined as described below.

The temperature of the resultant solid particles 1 was increased from 25° C. to 600° C. at 10° C./min with a thermogravimetric differential thermal analyzer “TG-DTA EXSTAR 6200” manufactured by Hitachi High-Tech Science Corporation while air was supplied at 200 mL/min, followed by the measurement of the mass of the residue after the burning. A value calculated from the following equation by using the mass of the residue when only a raw material composition having the same mass as that of the raw material composition used in the production of the solid particles 1 was subjected to the same treatment was defined as a powder adhesion ratio. The result is shown in Table 3.

$$( Powder adhesion ratio ( mass \% ) ) = ( mass \% of residue of solid particles 1 ) - [ \text{} ( mass \% of residue of raw material composition ) / [ \text{} 100 - \text{} ( mass \% of residue of raw material composition ) ] ] \times [ \text{} 100 - ( mass \% of residue of solid particles 1 ) ]$$

### (5) Mass of Solid Particle

Ten particles were randomly sampled from the resultant solid particles 1, and their masses were measured. The number-average mass of the 10 particles is shown in Table 3.

### (6) Strength of Solid Particle

Ten particles were randomly sampled from the resultant solid particles 1, and the particles were crushed with MODULAR COMPACT RHEOMETER MCR-302 manufactured by Anton Paar Japan K.K. while parallel plates each having a diameter of 25 mm were lowered at a speed of 20 μm/s. The strength of each of the particles was measured by reading a collapse point from the values of a load and a displacement. The number-average strength of the 10 particles is shown in Table 3.

### (7) Observation of Section

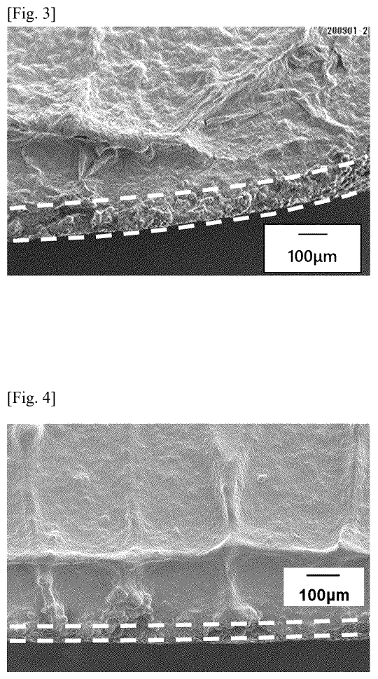

Two particles were randomly sampled from the resultant solid particles 1, and each of the solid particles 1 was cut with a knife, followed by the observation of a section thereof with a scanning electron microscope VE-7800 manufactured by Keyence Corporation. Images of 4 sites at the top and bottom, and left and right of the section of each particle were taken, and the thickness of a shell portion formed of the raw material composition having incorporated thereinto the powder, the shell portion coating the surface of a core portion, was measured at 3 sites for each of the taken images, that is, at a total of 12 sites for each particle. The average of the thicknesses of the shell portions at a total of 24 sites was determined, and was shown in Table 3. In addition, as shown in FIG. 3, the powder not only adhered to the surface of each of the solid particles 1 but also was incorporated into the raw material composition in a thickness of from about 100 μm to about 200 μm from the surface of the core portion.

### Examples 2 to 5

The solid particles 1 for lipsticks were each produced in the same manner as in Example 1 except that the respective conditions of the production method were changed as shown in Table 3. Results obtained by evaluating the resultant solid particles 1 in the same manner as in Example 1 are shown in Table 3.

### Example 6

The solid particles 1 for a lipstick were produced in the same manner as in Example 1 except that: the vibration device 15 and the trough 16 used in Example 1 were changed to the bowl feeder 20 (PARTS FEEDER ER-30BR manufactured by Sinfonia Technology Co., Ltd.) including the vibration device 15 and the bowl 18; and the respective conditions of the production method were changed as shown in Table 3. Results obtained by evaluating the resultant solid particles 1 in the same manner as in Example 1 are shown in Table 3. The contact time in Example 6 is a time period required for the granular raw materials 13 to which the powder 14 has adhered to reach the outlet of the bowl 18 after the dropping of the granular raw materials 13.

### Example 7

The solid particles 1 for a lipstick were produced in the same manner as in Example 1 except that the powder 14 was changed to the mixture (spherical silica: pigment=9:1) of spherical silica, which had an average particle diameter D50 of 15 μm and an oil absorption of 150 mL/100 g, and a pigment (METASHINE MT1080TY manufactured by Nippon Sheet Glass Company, Limited). Results obtained by evaluating the resultant solid particles 1 in the same manner as in Example 1 are shown in Table 3.

### Example 8

The solid particles 1 for an eye shadow were produced in the same manner as in Example 1 except that the raw material composition was changed to the raw material composition for an eye shadow. Results obtained by evaluating the resultant solid particles 1 in the same manner as in Example 1 are shown in Table 3.

### Comparative Example 1

The solid particles 1 for a lipstick were produced in the same manner as in Example 1 except that a method of bringing the granular raw material and the powder into contact with each other was changed as follows: the granular raw materials 13 were dropped onto the powder 14, which was left at rest, under room temperature (25° C.); the powder was brought into a fluid state with stirring means formed of an anchor blade; and the powder 14 was caused to adhere to the surface of each of the granular raw materials 13 for 60 seconds, followed by natural cooling for 1 minute. Results obtained by evaluating the resultant solid particles 1 in the same manner as in Example 1 are shown in Table 3.

### Comparative Example 2 and Comparative Example 3

The powder 14 was spread all over a balance dish (BDA-3 manufactured by AS ONE Corporation) so as to have a thickness of 10 mm, and the granular raw materials 13 were dropped onto the powder 14 under room temperature (25° C.), followed by natural cooling for 1 minute. With regard to Comparative Example 2, all the contents of the balance dish were sieved, and with regard to Comparative Example 3, the particles were recovered with a spatula and sieved. The solid particles 1 for lipsticks were each produced in the same manner as in Example 1 except the foregoing. Results obtained by evaluating the resultant solid particles 1 in the same manner as in Example 1 are shown in Table 3.

TABLE 3

| | | | | Example | | | | | |
|---|---|---|---|---|---|---|---|---|---|
| | | | | 1 | 2 | 3 | 4 | 5 | 6 |
| Granular raw material | | Raw material | | Lipstick | Lipstick | Lipstick | Lipstick | Lipstick | Lipstick |
| | | composition | | | | | | | |
| | | Melting poi [symbol] | [° C.] | 70 | 70 | 70 | 70 | 70 | 70 |
| Powder | | Kind | | Silica | Silica | Silica | Silica | Silica | Silica |
| | | Average part | [μm] | 15 | 15 | 15 | 15 | 15 | 15 |
| Production condition | | Dropping | [° C.] | 90 | 90 | 90 | 90 | 90 | 90 |
| | | temperatur | | | | | | | |
| | | Dropping | [mm] | 117 | 41 | 41 | 41 | 41 | 41 |
| | | distance | | | | | | | |
| | | Apparatus | | Vibration | Vibration | Vibration | Vibration | Vibration | Bowl feeder |
| | | | | feeder | feeder | feeder | feeder | feeder | |
| | | Amplitude | [mm] | 0.663 | 0.487 | 0.663 | 1.214 | 0.617 | 0.709 |
| | | Frequency | [Hz] | 54 | 54 | 54 | 57 | 60 | 58 |
| | | Contact tim [symbol] | [sec] | 5 | 15 | 7 | 3 | 11 | 22 |
| Evaluation | Adhesion | Adhesion | [%] | 23 | 75 | 23 | 58 | 58 | 0 |
| | resistance | ratio | | | | | | | |
| | Sense of use*1 | | | Satisfactory | Satisfactory | Satisfactory | Satisfactory | Satisfactory | Satisfactory |
| | Transportation | Adhesion | [%] | 0 | 54 | 0 | 46 | 33 | 23 |
| | resistance | ratio | | | | | | | |
| | Powder | <Average | [mass %] | 8.3 | 8.0 | 9.0 | 8.6 | 8.4 | 11.6 |
| | adhesion ratio | for N = 3> | | | | | | | |
| | Solid particle | <Average | [mg] | 20 | 17 | 18 | 18 | 18 | 18 |
| | mass | for [symbol] | | | | | | | |
| | Solid particle | <Average | [N] | 0.17 | 0.19 | 0.21 | 0.23 | 0.19 | 0.23 |
| | strength | for [symbol] | | | | | | | |
| | Average | <Average | [mm2] | 11 | 11 | 11 | 11 | 11 | 12 |
| | projected area | for [symbol] | | | | | | | |
| | Average diameter | <Average | [mm] | 3.7 | 3.7 | 3.8 | 3.7 | 3.7 | 3.9 |
| | | for [symbol] | | | | | | | |
| | Average height | <Average | [mm] | 2.4 | 2.5 | 2.0 | 2.3 | 2.4 | 2.3 |
| | | for [symbol] | | | | | | | |
| | Average height/ | <Average | [%] | 64.9 | 65.4 | 53.1 | 61.5 | 64.0 | 57.5 |
| | Average diameter | for [symbol] | | | | | | | |
| | Shell portion | <Average | [μm] | 136 | 122 | 120 | 136 | 125 | 182 |
| | thickness*2 | for [symbol] | | | | | | | |

| | | | | Example | | Comparative Example | | |
|---|---|---|---|---|---|---|---|---|
| | | | | 7 | 8 | 1 | 2 | 3 |
| Granular raw material | | Raw material | | Lipstick | Eye | Lipstick | Lipstick | Lipstick |
| | | composition | | | shadow | | | |
| | | Melting poi [symbol] | [° C.] | 70 | 79 | 70 | 70 | 70 |
| Powder | | Kind | | Silica + | Silica | Silica | Silica | Silica |
| | | | | pigment | | | | |
| | | Average part | [μm] | 15 | 15 | 15 | 15 | 15 |
| Production condition | | Dropping | [° C.] | 90 | 80 | 90 | 90 | 90 |
| | | temperatur | | | | | | |
| | | Dropping | [mm] | 41 | 41 | — | — | — |
| | | distance | | | | | | |
| | | Apparatus | | Vibration | Vibration | Stirring | — | — |
| | | | | feeder | feeder | tank | | |
| | | Amplitude | [mm] | 0.663 | 0.663 | — | — | — |
| | | Frequency | [Hz] | 54 | 54 | — | — | — |
| | | Contact tim [symbol] | [sec] | 7 | 7 | 60 | 60 | 60 |
| Evaluation | Adhesion | Adhesion | [%] | 25 | 0 | 117 | 117 | 115 |
| | resistance | ratio | | | | | | |

| Sense of use*1 | | | Satisfactory | Satisfactory | Satisfactory | Satisfactory | Satisfactory |
|---|---|---|---|---|---|---|---|
| Transportation | Adhesion | [%] | 38 | 46 | 92 | 86 | 100 |
| resistance | ratio | | | | | | |
| Powder | <Average | [mass %] | 9.2 | 8.2 | 6.9 | 4.8 | 3.8 |
| adhesion ratio | for N = 3> | | | | | | |
| Solid particle | <Average | [mg] | 18 | 21 | 19 | 20 | 19 |
| mass | for [symbol] | | | | | | |
| Solid particle | <Average | [N] | 0.21 | 0.18 | 0.13 | 0.23 | 0.20 |
| strength | for [symbol] | | | | | | |
| Average | <Average | [mm2] | 12 | 11 | 14 | 9 | 12 |
| projected area | for [symbol] | | | | | | |
| Average diameter | <Average | [mm] | 3.9 | 3.8 | 4.1 | 3.5 | 3.9 |
| | for [symbol] | | | | | | |
| Average height | <Average | [mm] | 2.1 | 2.3 | 2.8 | 2.6 | 2.6 |
| | for [symbol] | | | | | | |
| Average height/ | <Average | [%] | 54.8 | 60.7 | 66.4 | 75.5 | 66.4 |
| Average diameter | for [symbol] | | | | | | |
| Shell portion | <Average | [μm] | 155 | 152 | 102 | 50 | 45 |
| thickness*2 | for [symbol] | | | | | | |

*1Particles free of roughness were judged to be “satisfactory”, and particles having roughness were judged to be “unsatisfactory”.
*2The thickness means the thickness of a raw material composition having incorporated thereinto powder. [symbol]
indicates data missing or illegible when filed

As shown in Table 3, the solid particles 1 of Examples and the solid particles of Comparative Examples each had a satisfactory sense of use because the core portion of each of the particles formed of the raw material composition containing the oily components was coated with the shell portion thereof containing the silica serving as the powder. In addition, the solid particles 1 of Examples each had satisfactory adhesion resistance and satisfactory transportation resistance. Meanwhile, none of the solid particles of Comparative Examples had sufficient adhesion resistance and sufficient transportation resistance because the average strengths of the particles were low. This is probably because in each of the solid particles 1 of Examples, the silica adhered to the surface of the particle, and moreover, as shown in Table 3 and FIG. 3, the silica was incorporated into the raw material composition at a high density and in a uniform thickness to a depth of from about 110 μm to about 200 μm from the surface of the core portion thereof. Meanwhile, it was found that in each of the solid particles of Comparative Examples, as shown in FIG. 4, the silica adhering to the surface of the particle did not have any uniform thickness, and a portion substantially free of silica adhering thereto was present.

### DESCRIPTION OF THE REFERENCE NUMERALS

1: a solid particle
10: a raw material composition
11: a pump
12: a nozzle
13: a granular raw material
14: a powder
15: a vibration device
16: a trough
17: a sieve 17
18: a bowl 18
19: a vibration feeder
20: a bowl feeder

1. A method of producing a solid particle, comprising the following:
heating a raw material composition containing an oily component to impart fluidity thereto;
granulating the raw material composition having imparted thereto the fluidity to provide a granular raw material; and
bringing the granular raw material and a powder into contact with each other while vibration is applied to the powder, to thereby coat at least a part of a surface of the granular raw material with the powder.

2. The method of producing a solid particle according to claim 1, wherein the powder contains a pigment.

3. The method of producing a solid particle according to claim 1, wherein the powder contains silica.

4. The method of producing a solid particle according to claim 1, further comprising cooling the granular raw material.

5. The method of producing a solid particle according to claim 1, further comprising removing the powder that is free from adhering to the solid particle.

6. The method of producing a solid particle according to claim 1, further comprising heating the raw material composition to a melting point thereof or more prior to granulating the raw material composition to obtain the granular raw material.

7. The method of producing a solid particle according to claim 1, wherein the vibration has an amplitude of 0.3 mm or more and a frequency of 30 Hz or more.

8. The method of producing a solid particle according to claim 1, wherein the powder has an average particle diameter D₅₀ of 0.01 μm or more and 500 μm or less.

9. The method of producing a solid particle according to claim 1, wherein the solid particles have an average mass per particle of 1 mg or more and 10,000 mg or less.

10. A solid particle having a core-shell structure, comprising:
a core portion formed of a raw material composition; and
a shell portion configured to coat at least part of a surface of the core portion,
wherein the shell portion is formed of the raw material composition having incorporated thereinto a powder, and the raw material composition of the shell portion having incorporated thereinto the powder has a thickness of 110 μm or more.

11. A cosmetic, comprising a solid particle having a core-shell structure, comprising:
a core portion formed of a raw material composition; and
a shell portion configured to coat at least part of a surface of the core portion,
wherein the shell portion is formed of the raw material composition having incorporated thereinto a powder, and the raw material composition having incorporated thereinto the powder has a thickness of 110 μm or more.

12. The solid particle according to claim 10, wherein the powder contains a pigment.

13. The solid particle according to claim 10, wherein the powder contains silica.

14. The solid particle according to claim 10, wherein the powder has an average particle diameter D₅₀ of 0.01 μm or more and 500 μm or less.

15. The solid particle according to claim 10, wherein the solid particles have an average mass per particle of 1 mg or more and 10,000 mg or less.

16. The cosmetic according to claim 11, wherein the powder contains a pigment.

17. The cosmetic according to claim 11, wherein the powder contains silica.

18. The cosmetic according to claim 11, wherein the powder has an average particle diameter D₅₀ of 0.01 μm or more and 500 μm or less.

19. The cosmetic according to claim 11, wherein the solid particles have an average mass per particle of 1 mg or more and 10,000 mg or less.

20. The cosmetic according to claim 11, further comprising a cosmetic palette, wherein the solid particle is mounted on the cosmetic palette.
